(19) United States
(12) Patent Application Publication
(10) Pub. No.: US 2025/0186703 A1
(43) Pub. Date: Jun. 12, 2025

(54) PLUNGER ROD ASSEMBLY CONFIGURED TO REDUCE USE ERROR ASSOCIATED WITH OPERATION OF A MEDICAL DEVICE INCLUDING THE PLUNGER ROD ASSEMBLY

(71) Applicant: AKERO THERAPEUTICS, INC., South San Francisco, CA (US)

(72) Inventors: Valerie Fenster, South San Francisco, CA (US); Catherine Tamura, South San Francisco, CA (US); Timothy P. Rolph, Brookline, MA (US)

(21) Appl. No.: 18/974,338

(22) Filed: Dec. 9, 2024

Related U.S. Application Data
(60) Provisional application No. 63/608,029, filed on Dec. 8, 2023.

(51) Int. Cl. A61M 5/31

(52) U.S. Cl. CPC A61M 5/3137; A61M 2205/586

(57) ABSTRACT

A plunger rod assembly for use with a medical device. The plunger rod assembly includes a finger rest adapted to be coupled to a barrel of the medical device, and a plunger rod coupled to the finger rest. The finger rest includes an interface that selectively engages the plunger rod to control movement of the plunger rod relative to the finger rest. The plunger rod includes a first locking mechanism and a second locking mechanism. When the plunger rod is in a first position, the first locking mechanism engages a first portion of the interface such that the plunger rod is only movable relative to the finger rest by moving the plunger rod in an axial direction until the plunger rod reaches a second position. When the plunger rod is in the second position, the second locking mechanism engages a second portion of the interface such that the plunger rod is only movable relative to the finger rest by rotating the plunger rod in a first radial direction until the plunger rod reaches a third position. When the plunger rod is in the third position, the second locking mechanism engages the first portion of the interface such that the plunger rod is only movable relative to the finger rest by moving the plunger rod in the axial direction until the plunger rod reaches an activated position.

### FIELD OF THE DISCLOSURE

The present disclosure generally relates to a plunger rod assembly, and, more particularly, to a plunger rod assembly that is for use with a medical device (e.g., a syringe-based device) and includes two locking mechanisms designed to control movement of the plunger rod so as to reduce use error associated with operation of the injection device including the plunger rod assembly.

### BACKGROUND

Syringe-based medical devices are commonly used to inject one or more drug products (or other fluids) into a patient. In some cases, a medical professional (e.g., a doctor) will be responsible for preparing and/or utilizing the syringe-based medical device (syringe) to inject the one or more drug products into the patient. In other cases, in addition to the medical professional, the patient or patient's caregiver may also be responsible for self-preparing the syringe-based medical device and self-administering the one or more drug products utilizing the syringe. For example, one known syringe-based medical device is a dual chamber syringe that requires the user to reconstitute a powdered form of the drug product in the first chamber with a liquid solution in the second chamber, before the user can expel the reconstituted drug from the syringe by depressing the plunger rod of the syringe. Some syringe-based medical devices may require three plunger rod movements: (1) a first movement to push the plunger rod to mix the contents of the one or more chambers (reconstitution), (2) a second movement of the plunger rod to enable the syringe to allow the drug product to be expelled from the syringe (activation) after an appropriate reconstitution wait time (e.g., 10 minutes) to fully mix between the reconstitution and the injection, and (3) a third movement to push the plunger rod down to finally expel the drug product and proceed with the self-administration (injection).

### BRIEF DESCRIPTION OF THE DRAWINGS

The features of this disclosure which are believed to be novel are set forth with particularity in the appended claims. The present disclosure may be best understood by reference to the following description taken in conjunction with the accompanying drawings, in which like reference numerals identify like elements in the several figures, in which:

FIG. 1A is a perspective view of an example of a plunger rod assembly constructed in accordance with the teachings of the present disclosure and shown in use with a conventional syringe barrel, the plunger rod assembly including a finger rest and a plunger rod in a first position relative to the finger rest.

FIG. 1B is a plan view of the plunger rod assembly FIG. 1A.

FIG. 2A is a close-up view of the plunger rod of the plunger rod assembly of FIG. 1A.

FIG. 2B is an exploded view of the elements of the plunger rod assembly of FIG. 1A.

FIG. 2C is a close-up, perspective view of the finger rest of the plunger rod assembly of FIG. 1A.

FIG. 2D is a top view of the plunger rod assembly of FIG. 2C.

FIG. 2E is a perspective view of the syringe barrel of FIG. 1A.

FIG. 2F is a perspective view of a first stopper utilized in connection with the plunger rod assembly of FIG. 1A.

FIG. 2G is a perspective view of a second stopper utilized in connection with the plunger rod assembly of FIG. 1A.

FIG. 2H is a perspective view of the plunger rod assembly of FIG. 1A and a portion of the syringe barrel including the second stopper.

FIG. 3A is a close-up, perspective view of the plunger rod assembly of FIG. 1A, showing the engagement between the plunger rod and the finger rest of the plunger rod assembly when the plunger rod is in the first position.

FIG. 3B is another close-up, perspective view similar to FIG. 3A.

FIG. 3C is a perspective view of the plunger rod assembly of FIG. 1A, showing the movement of the plunger rod from the first position to a second position relative to the finger rest.

FIG. 3D is a close-up view of a portion of FIG. 3C.

FIG. 4A is a perspective view showing the plunger rod in the second position.

FIG. 4B is a top view of FIG. 4A.

FIG. 4C is a plan view of FIG. 4A.

FIG. 4D is similar to FIG. 4C but shows the position of the plunger rod within the syringe barrel.

FIG. 4E is a close-up, perspective view of the plunger rod assembly of FIG. 1A, showing the engagement between the plunger rod and the finger rest of the plunger rod assembly when the plunger rod is in the second position.

FIG. 4F is another close-up, perspective view similar to FIG. 4E.

FIG. 5A is a top view illustrating the plunger rod rotated 90 degrees in a clockwise direction from the second position to a third position relative to the finger rest.

FIG. 5B is a close-up, perspective view of the plunger rod assembly of FIG. 1A, showing the engagement between the plunger rod and the finger rest of the plunger rod assembly when the plunger rod is in the third position.

FIG. 5C is a cross-sectional view of the plunger rod and the finger rest when the plunger rod is in the third position shown in FIGS. 5A and 5B.

FIG. 6A is a perspective view of the plunger rod assembly of FIG. 1A, showing the plunger rod in a fourth, activated position relative to the finger rest.

FIG. 6B is a plan view of FIG. 6A.

FIG. 7 illustrates another example of a plunger rod assembly constructed in accordance with the teachings of the present disclosure and shown in use with a conventional syringe, the plunger rod assembly including a finger rest and a plunger rod in a first position relative to the finger rest.

FIG. 8A is a plan view of the plunger rod assembly of FIG. 7 but showing the plunger rod in a second position relative to the finger rest.

FIG. 8B is a top view of the plunger rod assembly of FIG. 8A.

FIG. 9 is a top view of the plunger rod assembly of FIG. 7 but showing the plunger rod in a third position relative to the finger rest.

FIG. 10A is a perspective view of the plunger rod assembly of FIGS. 8A and 8B.

FIG. 10B is a plan view of the plunger rod assembly of FIG. 10A.

### SUMMARY

A plunger rod assembly for use with a medical device. The plunger rod assembly includes a finger rest adapted to be coupled to a barrel of the medical device, and a plunger rod coupled to the finger rest. The finger rest includes an interface that selectively engages the plunger rod to control movement of the plunger rod relative to the finger rest. The plunger rod includes a first locking mechanism and a second locking mechanism. When the plunger rod is in a first position, the first locking mechanism engages a first portion of the interface such that the plunger rod is only movable relative to the finger rest by moving the plunger rod in an axial direction until the plunger rod reaches a second position. When the plunger rod is in the second position, the second locking mechanism engages a second portion of the interface such that the plunger rod is only movable relative to the finger rest by rotating the plunger rod in a first radial direction until the plunger rod reaches a third position. When the plunger rod is in the third position, the second locking mechanism engages the first portion of the interface such that the plunger rod is only movable relative to the finger rest by moving the plunger rod in the axial direction until the plunger rod reaches an activated position.

### DETAILED DESCRIPTION

Known syringe-based medical devices such as the dual chamber syringe discussed above are prone to use-related errors during operation, typically by a user (e.g., the medical professional, the patient or the patient's caregiver) responsible for self-preparing and/or self-administering one or more drug products (or other fluids) utilizing the syringe-based device. For example, many known syringe-based medical devices allow the user to inadvertently activate a syringe before reconstitution has occurred, such that the user is able to expel undissolved or partially dissolved drug product through the end of the syringe-based device, resulting in a missed dose or delivery of a partial dose. Many of these known syringe-based medical devices may alternatively or additionally allow the user to misalign components of the syringe-based medical device during activation, typically due to over-rotation or under-rotation of the plunger rod, thereby preventing expulsion of the drug product from the medical syringe-based device and causing user frustration, delayed therapy and wasted needles. Many known syringe-based medical devices also allow the user to inadvertently unscrew the plunger rod from the syringe-based device by turning the plunger rod counter-clockwise out of the stopper, disconnecting the plunger rod from the syringe-based medical device. This action not only causes user frustration but, more problematically, can lead to delayed therapy if drug product is lost from the syringe barrel and no other syringes are available.

The present disclosure is directed to a plunger rod assembly that aims to address these and other limitations or use-related errors with known syringe-based medical devices and includes a plunger rod and a finger rest that interacts with the plunger rod to achieve the intended functionality the first time the user self-prepares and self-administers drug product(s) contained within the syringe barrel (barrel) with this plunger rod assembly. The disclosed plunger rod assembly generally allows a user to self-prepare and self-administer drugs but is configured to significantly reduce use-related error associated with the self-preparation and self-administration. More particularly, the disclosed plunger rod assembly is configured to prevent inadvertent activation of the plunger rod (e.g., before the drug product has been properly reconstituted), inaccurate activation of the plunger rod, and/or inadvertent removal of the plunger rod from the rest of the medical device. More accurate activation also eliminates the need to lift the medical device (and inadvertently lock any passive or automatically deployed safety guard needles coupled thereto) off the skin to re-align the plunger rod and reduces the frequency of needing to replace the passive safety needle before self-administering or injecting the drug. This reduces user frustration, as well as the number of passive safety needles that are typically wasted, leading to achieving the intended syringe functionality.

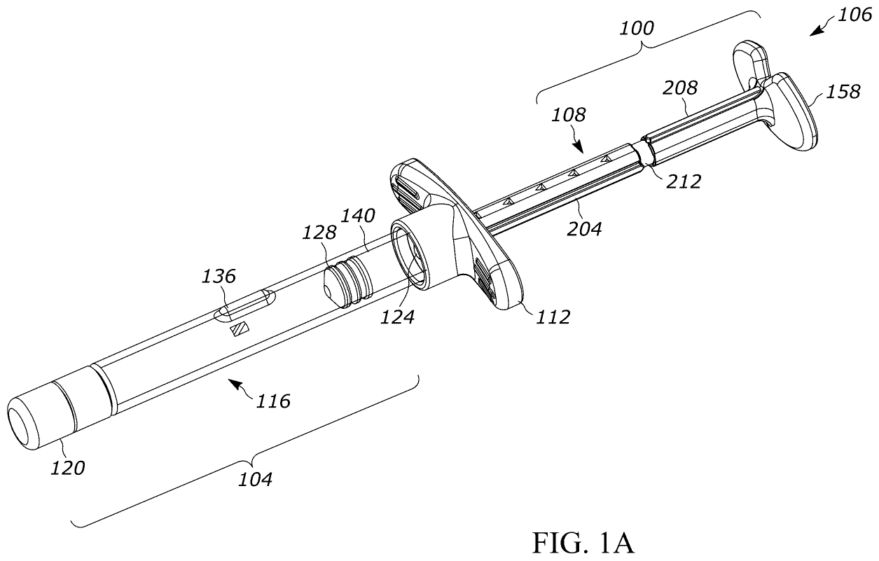

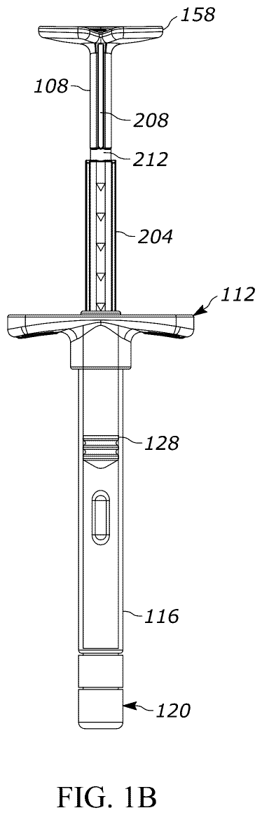

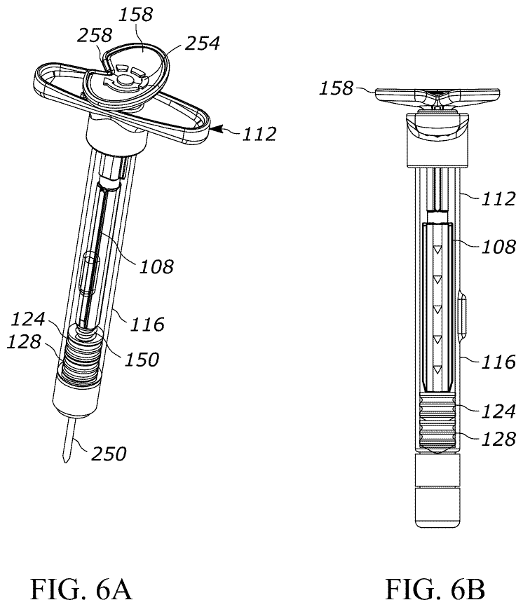

FIGS. 1A-6B illustrate one example of a plunger rod assembly 100 that is constructed in accordance with the teachings of the present disclosure and is shown in use with a conventional barrel assembly 104 to form a syringe-based medical device 106. The plunger rod assembly 100 generally includes a plunger rod 108 and a finger rest 112 (which may also be referred to herein as a gripping element) for a user to grip the plunger rod assembly 100). The barrel assembly 104 includes a barrel 116 for receiving the plunger rod 108 and to which the finger rest 112 can be coupled, as well as a cap or tamper-evident seal 120, first and second stoppers 124, 128 movably disposed in the barrel 116, and a bypass valve. The barrel 116 has two chambers 136, 140. The first chamber 136 can be pre-filled with a solid (e.g., a powder such as a drug product in a dry powder form) and the second chamber 140 can be pre-filled with a diluent (e.g., water for injection or an appropriate liquid formulation). The bypass valve is disposed in the barrel 116 and helps to direct the contents of the second chamber 140 (e.g., the diluent) into the first chamber 136 when desired. The cap or tamper evident seal 120 ensures that the sterile container closure assembly of the aperture at the end of the barrel 116, which usually features a luer-fixing for attaching a passive safety needle 250 (see FIG. 6A), remains intact. The plunger rod assembly 100 can, however, be utilized with any number of different barrel assemblies, e.g., different barrel assemblies having two (or more) chambers, barrel assemblies including only a single chamber, etc. The plunger rod assembly 100 can also be utilized as part of other medical devices (e.g., other injection devices such as pen injectors). Additionally, the first chamber 136 of the barrel 116 can also include a liquid (e.g., a drug product in liquid form) instead of the solid. Further, in other examples, the barrel assembly 104 can include two or more bypass valves disposed in the barrel 116.

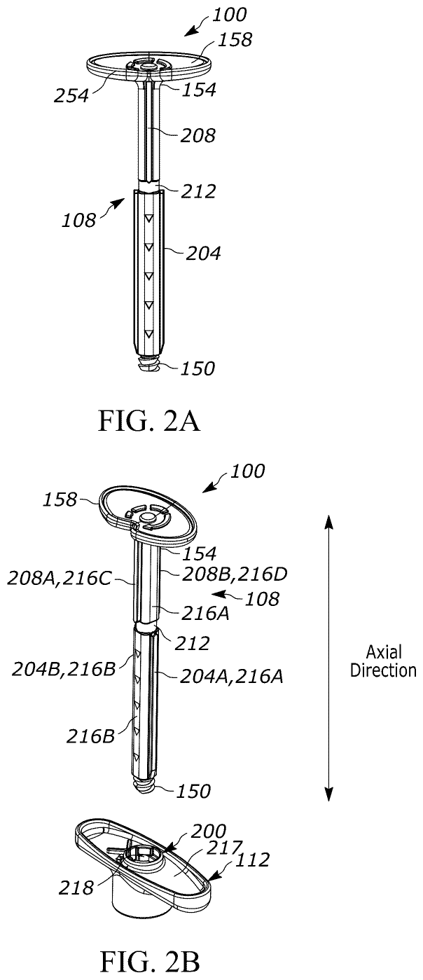

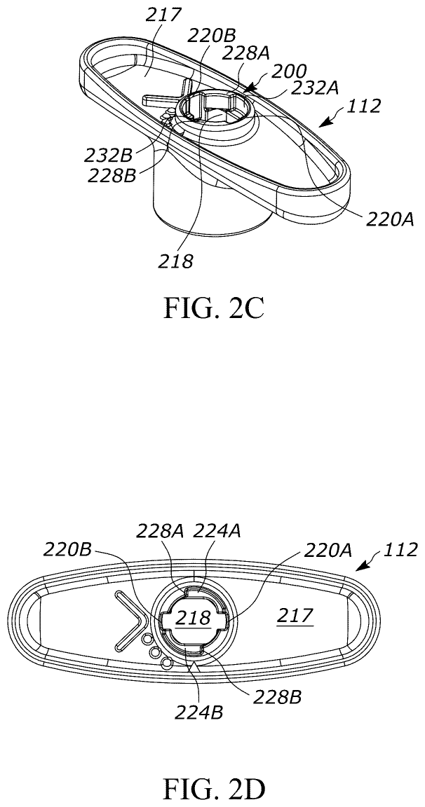

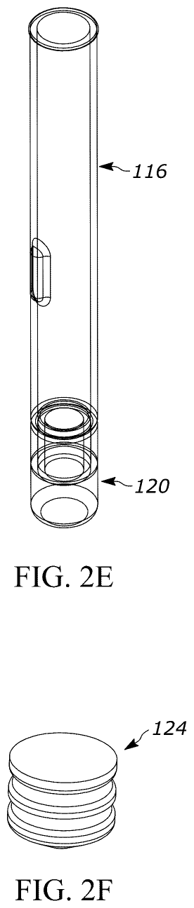

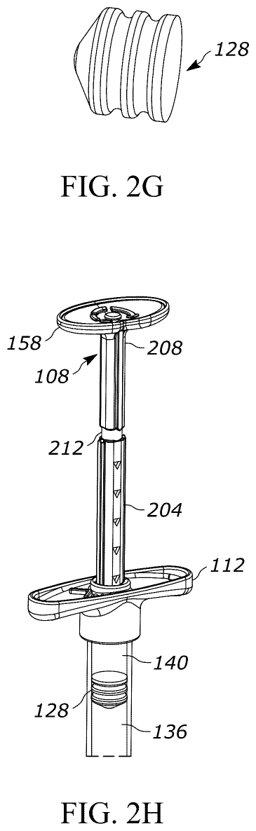

As best illustrated in FIGS. 2A and 2B, the plunger rod 108 has a first, distal end 150 and a second, proximal end 154 opposite the first end 150. When the plunger rod assembly 100 is coupled to the barrel 116, the first end 150 of the plunger rod 108 is connected to the first stopper 124 (which is initially disposed in the finger rest and therefore not visible in FIG. 1A), such that the first end 150 of the plunger rod 108 is movably located within the barrel 116. Meanwhile, the second, proximal end 154 of the plunger rod 108 is disposed outside of the barrel 116. The plunger rod assembly 100 also has a pad 158 (which can be referred to as a thumb pad) that is located at the proximal end 154 of the plunger rod 108 and allows the user to selectively move the plunger rod 108 by applying pressure.

Importantly, the plunger rod 108 and the finger rest 112 are designed and configured to interact with one another so as to achieve the benefits discussed above. For example, the plunger rod 108 and the finger rest 112 help to ensure the proper use and the correct order of operation of the medical device 106 by the user while the plunger rod assembly 100 is operatively connected to the barrel 116. The thumb pad and finger rest 112 also help the user to exert the necessary pressure on the plunger rod 108 and to operate the medical device 106.

To this end, in this example the finger rest 112 includes an interface 200 and the plunger rod 108 includes two locking mechanisms-a first, anti-rotational, locking mechanism 204 and a second, one-way, locking mechanism 208-as well as a recessed portion 212 that selectively engage with the interface 200 of the finger rest 112 to control axial and radial movement of the plunger rod 108 relative to the finger rest 112. More particularly, the first, anti-rotational, locking mechanism 204 selectively engages the interface 200 of the finger rest 112 to enable not only the transfer of diluent from the second chamber 140 into the first chamber 136 (which in this example contains dry powder), but also to prevent the inadvertent activation of the plunger rod 108. Meanwhile, the second, one-way, locking mechanism 208 that selectively engages with the interface 200 of the finger rest 112 to prevent the inaccurate activation of the plunger rod 108 as well as the inadvertent removal of the plunger rod 108 from the finger rest 112 and the barrel 116. Moreover, the second, one-way, locking mechanism 208 can be altered to provide more or less headspace within the barrel 116 during reconstitution/mixing as needed. For example, and beneficially, the second, one-way locking mechanism 208 can be lengthened so that the plunger rod 108 does not fully extend into the barrel 116 during reconstitution/mixing, which in turn provides additional headspace within the barrel 116 and beneficially reduces leakage from the barrel 116 during operation of the plunger rod assembly 108. Finally, the recessed portion 212 axially separates the first locking mechanism 204 from the second locking mechanism 208 to facilitate controlled movement of the plunger rod 108 (and the first and second locking mechanisms 204, 208) relative to the finger rest 112 (and the interface 200).

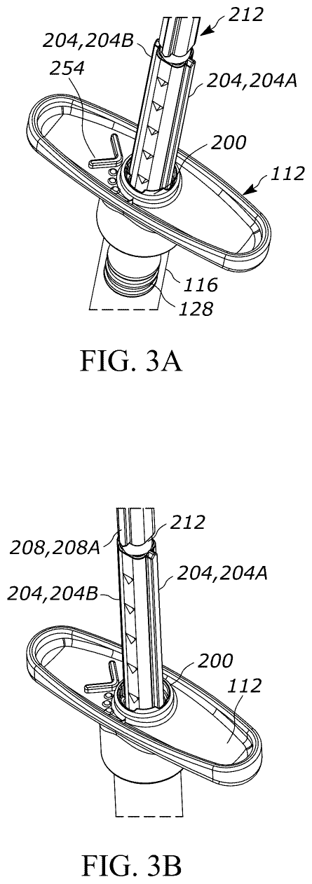

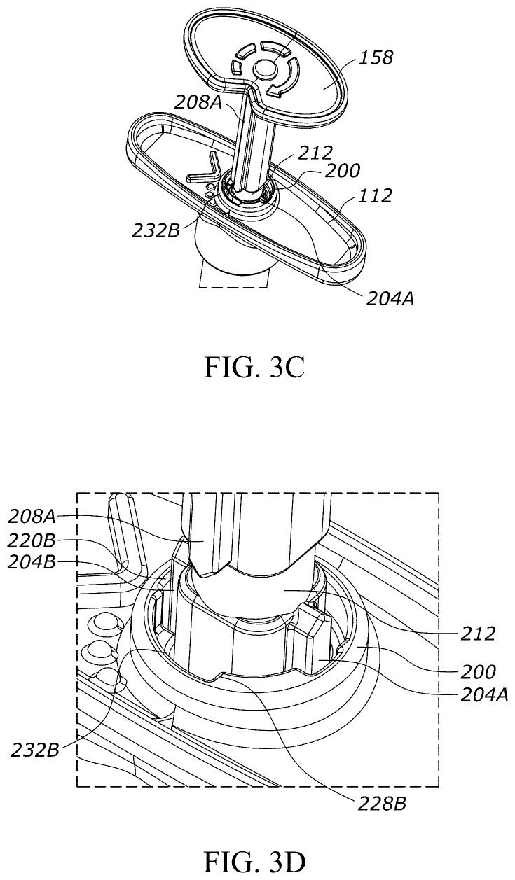

In this example, the first locking mechanism 204 takes the form of a pair of first projections 204A, 204B that extend outward from opposite sides of the plunger rod 108, respectively. More particularly, the first projection 204A extends radially outward from a first side 216A of the plunger rod 108, whereas the first projection 204B extends radially outward from a second side 216B of the plunger rod 108 opposite the first side 216A, as best illustrated in FIGS. 2B and 3A. In this example, each of the first projections 204A, 204B is a thin projection that extends in an axial direction between a position adjacent the distal end 150 of the plunger rod 108 and the recessed portion 212. In other examples, however, the first projections 204A, 204B can be positioned elsewhere along the plunger rod 108. In yet other examples, the first locking mechanism 204 can take the form of a single projection, more than two projections, or one or more different locking elements (e.g., tabs, ribs, latches). Further yet, the first locking mechanism 204 can be part of the finger rest 112 instead of being part of the plunger rod 108.

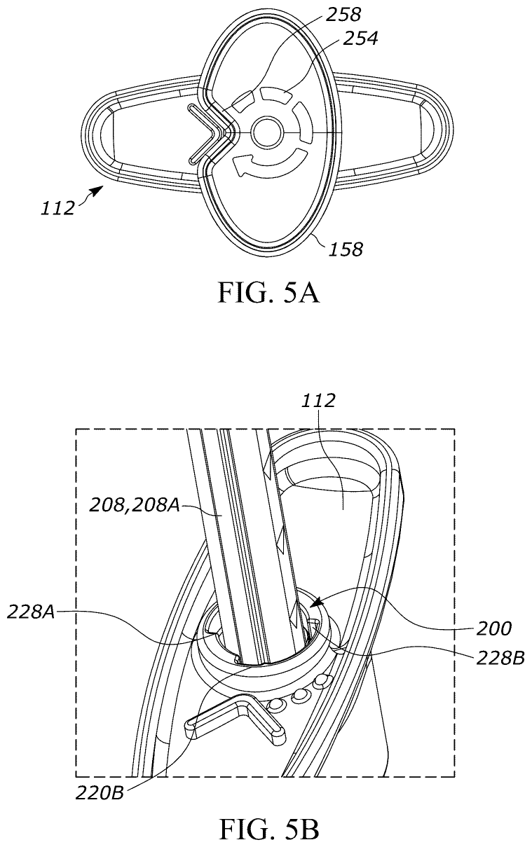

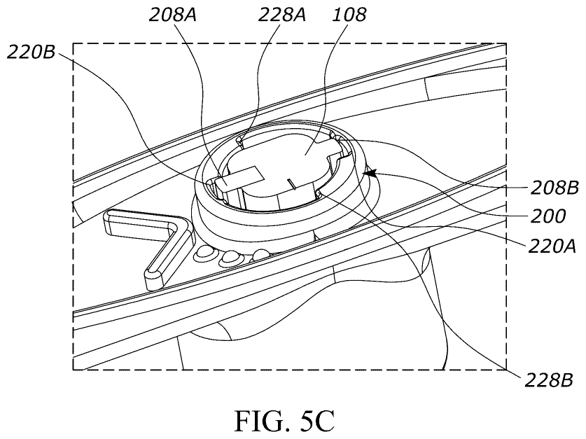

Similarly, in this example, the second locking mechanism 208 takes the form of a pair of second projections 208A, 208B (which may also be referred to as a rib) that extend outward from opposite sides of the plunger rod located between the first projections 204A, 204B. More particularly, the second projection 208A extends radially outward from a third side 216C of the plunger rod 108, whereas the second projection 208B extends radially outward from a fourth side 216D of the plunger rod 108 opposite the third side 216C, as best illustrated in FIGS. 2B and 5C. In turn, the second projections 208A, 208B are angularly offset from the first projections 204A, 204B, preferably by an angle equal to approximately 90 degrees. In other words, the second projection 208A is angularly offset from the first projections 204, 204B such that the second projection 208A is angularly disposed between the first projections 204, 204B. In this example, each of the second projections 208A, 208B is a thin projection that, like each of the first projections 204A, 204B, extends in the axial direction between the recessed portion 212 and the pad 158. In other examples, however, the second projections 208A, 208B can be positioned elsewhere along the plunger rod 108. In yet other examples, the second locking mechanism 208 can take the form of a single projection, more than two projections, or one or more different locking elements (e.g., tabs, ribs, latches). Further yet, the second locking mechanism 208 can be part of the finger rest 112 instead of being part of the plunger rod 108.

The interface 200 is an interior part of the finger rest 112. The interface 200 is coupled to (e.g., integrally formed with) an interior portion of a base 217 of the finger rest 112 and is configured to slidably receive and retain the plunger rod 108. More particularly, the interface 200 is configured to selectively engage the first and second locking mechanisms 204, 208 and the recessed portion 212 of the plunger rod 108 to provide the functionality described herein. In this example, the interface 200 includes a central aperture 218, first and second cutouts 220A, 220B, first and second shelves 224A, 224B, first and second stop surfaces 228A, 228B, and first and second grooves 232A, 232B that each define a travel path for the plunger rod 108 corresponding to the permissible rotation for the plunger rod 108 in operation, as best illustrated in FIGS. 2B-2D. In other examples, however, the interface 200 can include more or less components. In yet other examples, the interface 200 (and the components of the interface 200) can be part of the plunger rod 108 instead of the finger rest 112.

With continued reference to FIGS. 2B-2D, the central aperture 218 is sized to slidably receive and retain the plunger rod 108 as the plunger rod 108 is moved between different positions relative to the finger rest 112, as will be discussed in greater detail below. Each of the cutouts 220A, 220B is an annular, pocket like cutout formed in the interface 220 of the finger rest 112 at a position immediately radially outward of the central aperture 218. The first cutout 220A is formed in a first portion of the interface 200, while the second cutout 220B is formed in a second portion of the interface 200 opposite the first portion. In turn, at least in this example, the first and second cutouts 220A, 220B are disposed opposite and face one another, such that the first and second cutouts 220A, 220B correspond to rotation of the plunger rod 108 of approximately or exactly 90 degrees relative to the finger rest 112 at the appropriate time. Each of the first and second shelves 224A, 224B defines a curved surface that extends radially inwardly toward the central aperture 218. Each of the first and second shelves 224A, 224B is located between the first and second cutouts 220A, 220B, but with the first and second shelves 224A, 224B disposed on opposite sides of the interface 200. Similarly, each of the first and second stop surfaces 228A, 228B is located between the first and second cutouts 220A, 220B, but with the first and second stop surfaces 228A, 228B on opposite sides of the interface 220B. Finally, the first and second grooves 232A, 232B are annular grooves defined by and between the first and second cutouts 220A, 220B, respectively, the first and second shelves 224A, 224B, respectively, and the first and second stop surfaces 228A, 228B, respectively. More particularly, the first groove 232A is defined by and between the first stop surface 228A (which defines the starting point for the respective travel path), the first shelf 224A (which forms the bottom of the respective travel path), and the first cutout 220A (which defines the end point for the respective travel path). Likewise, the second groove 232B is defined by and between the second stop surface 228B (which defines the starting point for the respective travel path), the second shelf 224B (which forms the bottom of the respective travel path), and the second cutout 220B (which defines the end point for the respective travel path).

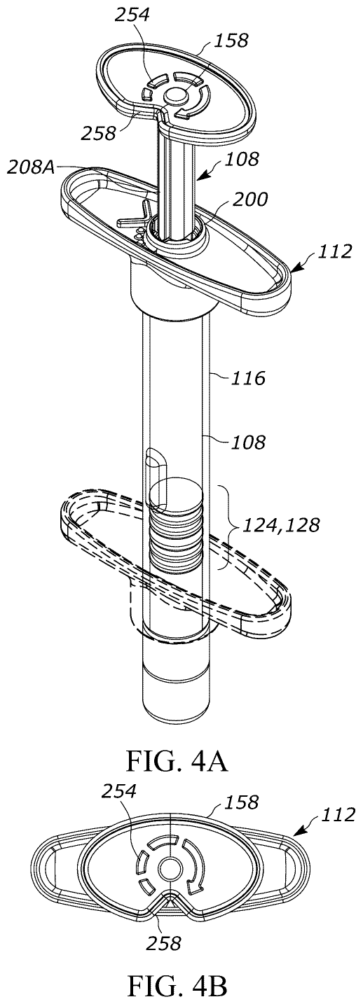

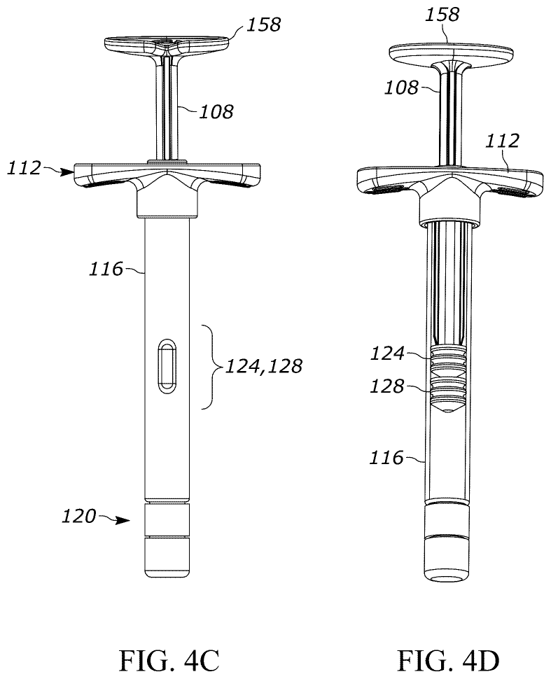

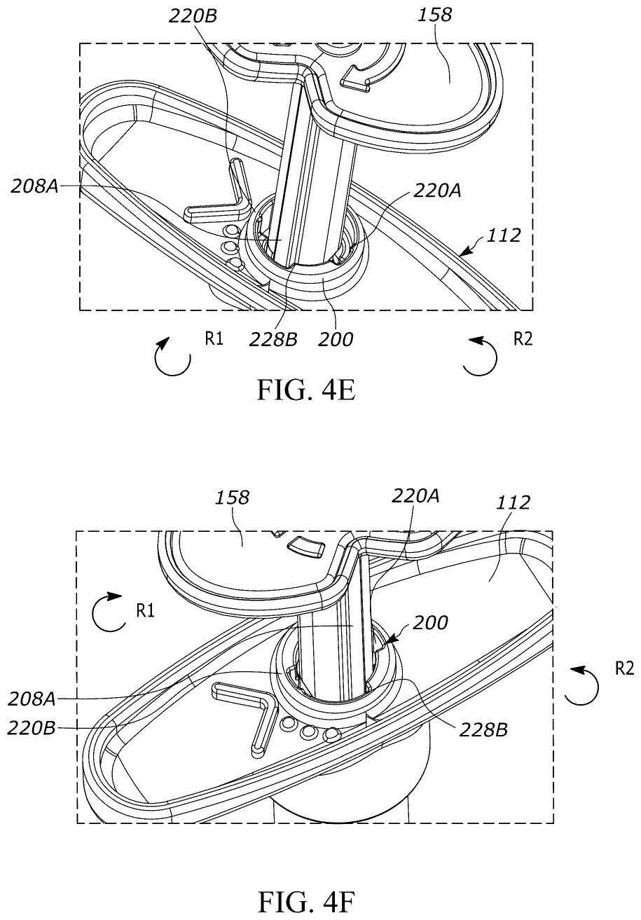

When the plunger rod assembly 100 is coupled to the barrel assembly 104 and is in operation, the plunger rod 108 is movable between at least four distinct positions relative to the finger rest 112—a first, initial or extended position (FIGS. 1A and 1B), a second, intermediate position (FIGS. 4A-4F), a third, intermediate position (FIGS. 5A-5C), and a fourth, final or activated position (FIGS. 6A and 6B). When the plunger rod 108 is in the first, initial or extended position best illustrated in FIGS. 1A and 1B, the first locking mechanism 204 engages a first portion of the interface 200. More particularly, the first projections 204A, 204B are disposed in the first and second cutouts 220A, 220B of the interface 200, respectively, and the second projections 208A, 208B are spaced from the first and second cutouts 220A, 220B, respectively. Because the first projections 204A, 204B are disposed in the first and second cutouts 220A, 220B, respectively, the plunger rod 108 cannot be rotated, thereby preventing inadvertent pre-activation prior to mixing of diluent from the second chamber 140 with dry powder in the first chamber 136 to reconstitute the liquid solution and preventing inadvertent removal of the plunger rod 108, which can be caused by the inadvertent disconnection of the plunger rod 108 from the first stopper 124 due to such rotation. Instead, and as illustrated in FIGS. 3A-3D, the user can only push the plunger rod 108 (and the first projections 204A, 204B) further into the barrel 116, which causes the first stopper 124 (connected to the plunger rod 108) to move towards the second stopper 128 (located approximately in the middle of the barrel 116) and causes the plunger rod 108 to push the diluent from the second chamber 140 of the barrel 116 into the first chamber 136 of the barrel 116 (which in this example contains dry power of a drug product), which in turn allows reconstitution of the dry powder as a solution in the first chamber 136. At this stage, the user cannot rotate the plunger rod 108 and can only move the plunger rod 108 axially relative to the finger rest 112 in the axial direction until the first locking mechanism 204 and the recessed portion 212 of the plunger rod 108 pass entirely through the interface 200 of the finger rest 112 and the second locking mechanism 208 passes through the first and second grooves 232A, 232B and engages the first and second shelves 224A, 224B of the interface (see FIGS. 4A-4F) to allow activation. In other words, the user cannot rotate the plunger rod 108 (inadvertently activate) until the plunger rod 108 reaches the second, intermediate position (and reconstitution has been allowed to occur).

Once the plunger rod 108 reaches this second, intermediate position (and reconstitution has presumably been allowed to occur), the first locking mechanism 204 no longer engages the first portion of the interface 200 and the second locking engagement 208 engages a second portion of the interface 200. More particularly, the first projections 204A, 204B are disposed outside of the cutouts 220A, 220B, respectively, and the second projections 208A, 208B engage the first and second shelves 224A, 224B, respectively (and, more particularly, a bottom of the projections 208A, 208B engage the first and second shelves 224A, 224B, respectively). As such, the user can only rotate the plunger rod 108 in a first radial direction R1 from the second, intermediate position to the third, intermediate position. In this example, the user can only rotate the plunger rod 108 ninety (90) degrees in the first radial direction R1, and the first radial direction R1 is the clockwise direction. Indeed, as best illustrated in FIGS. 4E and 4F, the first and second stops 228A, 228B are positioned to engage the second projections 208A, 208B of the second locking mechanism to prevent the plunger rod 108 from being rotated in a second radial direction R2 (which in this case is a counter-clockwise direction) opposite the first radial direction R1. Additionally, because the first and second shelves 224A, 224B engage the bottom of the projections 208A, 208B, respectively, the user is prevented from moving the plunger rod 108 axially relative to the finger rest 112 in the axial direction while the plunger rod 108 is in the second, intermediate position or is being rotated to the third, intermediate position.

As the user rotates the plunger rod 108 in the first radial direction, a bottom of each of the second projections 208A, 208B rotates through a respective one of the first and second grooves 232A, 232B and slides along a respective one of the shelves 224A, 224B of the interface 200 until the projections 208A, 208B are aligned with and disposed in the first and second cutouts 220A, 220B, respectively, as is illustrated in FIGS. 5A-5C, at which time the plunger rod 108 has reached the third, intermediate position. Once the plunger rod 108 reaches this third, intermediate position, the second locking mechanism 208 no longer engages the second portion of the interface 200 and instead engages the first portion of the interface 200 (and, more particularly, the first and second cutouts 220A, 220B). The engagement between the projections 208A, 208B of the second locking mechanism 208 and the first and second cutouts 220A, 220B, respectively, prevents further rotation of the plunger rod 108, either in the first radial (clockwise) direction R1 beyond 90 degrees or backwards in the second radial (counter-clockwise direction) R2, thereby preventing inaccurate activation of the plunger rod 108. Instead, the user can only push the plunger rod 108 axially further into the barrel 116 until the plunger rod 108 reaches the fourth, activated position shown in FIGS. 6A and 6B. In this activated position, the first and second stoppers 124, 128 are disposed at or near the end of the barrel 116 and the syringe-based medical device 106 is ready for the user to self-administer or inject the reconstituted drug product or medicine via the needle 250 attached to the end of the barrel 116 (e.g., which typically occurs prior to the initial mixing step).

The plunger rod assembly 100 can also include several design features that help guide the user through the process, as described in the instructions for use, of properly operating the syringe so as to fully reconstitute the drug, to activate the syringe properly at the appropriate time, and then to expel drug product as a liquid solution from the syringe. In this example, the design features take the form of markings 254 located on the pad 158 and the cut-out or indent 258 formed on the pad 158. These design features are arranged to indicate to the user how to properly move the plunger rod assembly from the second, intermediate position to the third, intermediate position and to the fourth, activated position shown in FIGS. 6A and 6B.

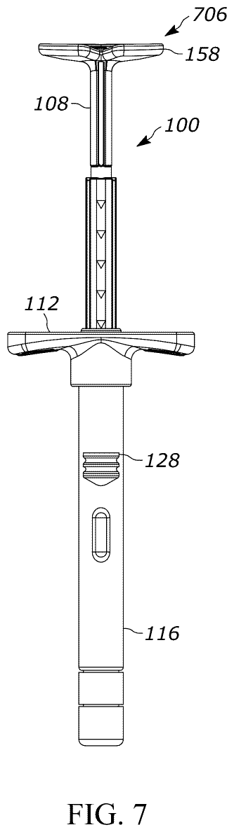

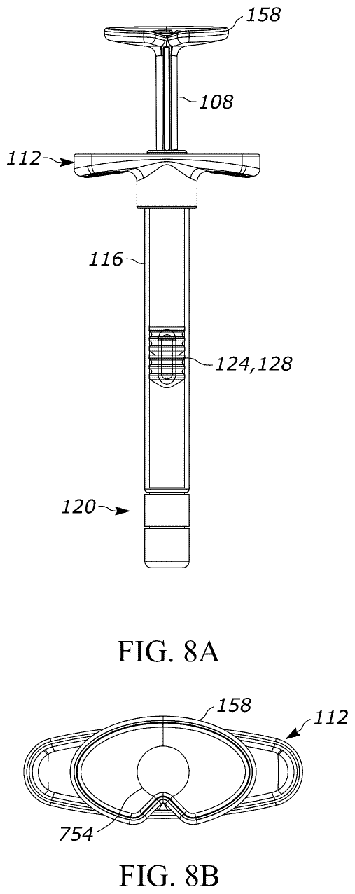

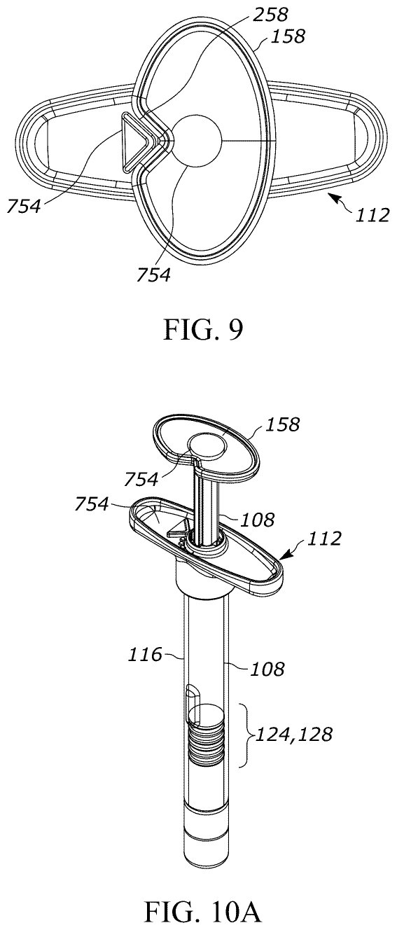

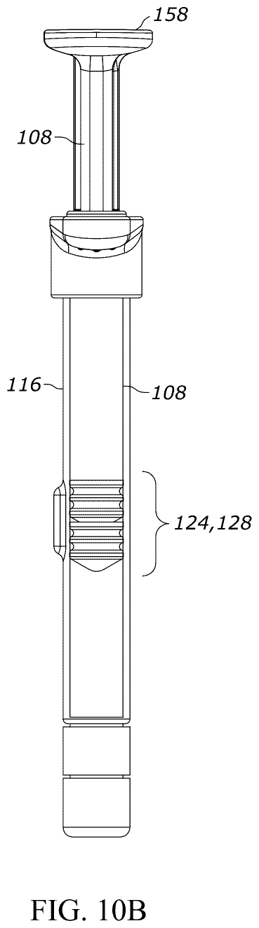

FIGS. 7-10B illustrate another example of a plunger rod assembly 700 that is constructed in accordance with the teachings and is shown in use with the conventional barrel assembly 104 to a syringe-based medical device 706. The plunger rod assembly 700 of FIGS. 7-10B is substantially similar to the plunger rod assembly 100 of FIGS. 1A-6B, with identical components depicted using identical reference numbers, and with the exception that the plunger rod assembly 700 includes different design features 754 than the plunger rod assembly 100.

Those skilled in the art will recognize that a wide variety of modifications, alterations, and combinations can be made with respect to the above-described embodiments without departing from the scope of the disclosure, and that such modifications, alterations, and combinations are to be viewed as being within the ambit of the inventive concept.

1. A plunger rod assembly for use with a medical device, the plunger rod assembly comprising:
a finger rest adapted to be coupled to a barrel of the medical device;
a plunger rod coupled to the finger rest,
wherein the finger rest includes an interface that selectively engages the plunger rod to control movement of the plunger rod relative to the finger rest,
wherein the plunger rod includes a first locking mechanism and a second locking mechanism,
wherein when the plunger rod is in a first position, the first locking mechanism engages a first portion of the interface such that the plunger rod is only movable relative to the finger rest by moving the plunger rod in an axial direction until the plunger rod reaches a second position,
wherein when the plunger rod is in the second position, the second locking mechanism engages a second portion of the interface such that the plunger rod is only movable relative to the finger rest by rotating the plunger rod in a first radial direction until the plunger rod reaches a third position, and
wherein when the plunger rod is in the third position, the second locking mechanism engages the first portion of the interface such that the plunger rod is only movable relative to the finger rest by moving the plunger rod in the axial direction until the plunger rod reaches an activated position.

2. The plunger rod assembly of claim 1, wherein the first locking mechanism comprises a pair of first projections that extend radially outward from opposite sides of the plunger rod.

3. The plunger rod assembly of claim 1, wherein the second locking mechanism comprises a pair of second projections that extend radially outward from opposite sides of the plunger rod.

4. The plunger rod assembly of claim 3, wherein the second projections are angularly offset from the first projections, respectively.

5. The plunger rod assembly of claim 1, wherein the plunger rod further comprises a recessed portion that axially separates the first locking mechanism from the second mechanism.

6. The plunger rod assembly of claim 1, wherein the first portion of the interface comprises a pair of cutouts, wherein the first locking mechanism is disposed in the pair of cutouts when the plunger rod is in the first position, and wherein the second locking mechanism is disposed in the pair of cutouts when the plunger rod is in the third position.

7. The plunger rod assembly of claim 6, wherein the first locking mechanism is spaced from the pair of cutouts when the plunger rod is in the second position and when the plunger rod is in the third position.

8. The plunger rod assembly of claim 6, wherein the second locking mechanism is spaced from the pair of cutouts when the plunger rod is in the first position and the plunger rod is in the second position.

9. The plunger rod assembly of claim 1, wherein the second portion of the interface comprises a pair of shelves, and wherein the second locking mechanism engages the pair of shelves when the plunger rod is in the second position.

10. The plunger rod assembly of claim 1, wherein the interface comprises a pair of stop surfaces, wherein when the plunger rod is in the second position, the stop surfaces are positioned to prevent rotation of the plunger rod in a second radial direction opposite the first radial direction.

11. The plunger rod assembly of claim 1, wherein the first radial direction is a clockwise direction.

12. A plunger rod assembly for use with a medical device, the plunger rod assembly comprising:
a finger rest adapted to be coupled to a barrel of the medical device;
a plunger rod coupled to the finger rest,
wherein the finger rest includes an interface that selectively engages the plunger rod to control movement of the plunger rod relative to the finger rest,
wherein the plunger rod includes a first locking mechanism and a second locking mechanism spaced from the first locking mechanism,
wherein the first locking mechanism comprises a pair of first projections that extend radially outward from opposite sides of the plunger rod,
wherein when the plunger rod is in a first position, the first projections engage a first portion of the interface such that the plunger rod is only movable relative to the finger rest by moving the plunger rod in an axial direction until the plunger rod reaches a second position,
wherein when the plunger rod is in the second position, the second locking mechanism engages a second portion of the interface such that the plunger rod is only movable relative to the finger rest by rotating the plunger rod in a first radial direction until the plunger rod reaches a third position, and
wherein when the plunger rod is in the third position, the second locking mechanism engages the first portion of the interface such that the plunger rod is only movable relative to the finger rest by moving the plunger rod in the axial direction until the plunger rod reaches an activated position.

13. The plunger rod assembly of claim 12, wherein the second locking mechanism comprises a pair of second projections that extend radially outward from opposite sides of the plunger rod.

14. The plunger rod assembly of claim 12, wherein the second projections are angularly offset from the first projections, respectively.

15. The plunger rod assembly of claim 12, wherein the plunger rod further comprises a recessed portion that axially separates the first locking mechanism from the second mechanism.

16. The plunger rod assembly of claim 12, wherein the first portion of the interface comprises a pair of cutouts, wherein the pair of first projections is disposed in the pair of cutouts, respectively, when the plunger rod is in the first position, and wherein the second locking mechanism is disposed in the pair of cutouts when the plunger rod is in the third position.

17. The plunger rod assembly of claim 16, wherein the pair of first projections is spaced from the pair of cutouts, respectively, when the plunger rod is in the second position and when the plunger rod is in the third position.

18. The plunger rod assembly of claim 16, wherein the second locking mechanism is spaced from the pair of cutouts when the plunger rod is in the first position and the plunger rod is in the second position.

19. The plunger rod assembly of claim 12, wherein the second portion of the interface comprises a pair of shelves, and wherein the second locking mechanism engages the pair of shelves when the plunger rod reaches the second position.

20. The plunger rod assembly of claim 12, wherein the interface comprises a pair of stop surfaces, wherein when the plunger rod is in the second position, the stop surfaces are positioned to prevent rotation of the plunger rod in a second radial direction opposite the first radial direction.

21. The plunger rod assembly of claim 12, wherein the first radial direction is a clockwise direction.

22. A plunger rod assembly for use with a medical device, the plunger rod assembly comprising:
a finger rest adapted to be coupled to a barrel of the medical device;
a plunger rod coupled to the finger rest,
wherein the finger rest includes an interface that selectively engages the plunger rod to control movement of the plunger rod relative to the finger rest,
wherein the plunger rod includes a first locking mechanism and a second locking mechanism spaced from the first locking mechanism,
wherein the first locking mechanism comprises a pair of first projections that extend radially outward from first and sides of the plunger rod,
wherein the second locking mechanism comprises a pair of second projections that extend radially outward from third and fourth sides of the plunger rod,
wherein when the plunger rod is in a first position, the first projections engage a first portion of the interface such that the plunger rod is only axially movable relative to the finger rest until the plunger rod reaches a second position,
wherein when the plunger rod is in the second position, the second projections engage a second portion of the interface such that the plunger rod is only rotatable relative to the finger rest until the plunger rod reaches a third position, and
wherein when the plunger rod is in the third position, the second projections engage the first portion of the interface such that the plunger rod is only axially movable relative to the finger rest until the plunger rod reaches an activated position.

23. The plunger rod assembly of claim 22, wherein the second projections are angularly offset from the first projections, respectively.

24. The plunger rod assembly of claim 22, wherein the plunger rod further comprises a recessed portion that axially separates the first locking mechanism from the second mechanism.

25. The plunger rod assembly of claim 22, wherein the first portion of the interface comprises a pair of cutouts, wherein the pair of first projections is disposed in the pair of cutouts, respectively, when the plunger rod is in the first position, and wherein the pair of second projections is disposed in the pair of cutouts, respectively, when the plunger rod is in the third position.

26. The plunger rod assembly of claim 25, wherein the pair of first projections is spaced from the pair of cutouts, respectively, when the plunger rod is in the second position and when the plunger rod is in the third position.

27. The plunger rod assembly of claim 25, wherein the pair of second projections is spaced from the pair of cutouts when the plunger rod is in the first position and the plunger rod is in the second position.

28. The plunger rod assembly of claim 22, wherein the second portion of the interface comprises a pair of shelves, and wherein the pair of second projections engage the pair of shelves, respectively, when the plunger rod reaches the second position.

29. The plunger rod assembly of claim 28, wherein the pair of second projections engage the pair of shelves as the plunger rod moves from the second position to the third position.

30. The plunger rod assembly of claim 22, wherein the interface comprises a pair of stop surfaces, wherein when the plunger rod is in the second position, the stop surfaces are positioned to prevent rotation of the plunger rod in a second radial direction opposite the first radial direction.

31. The plunger rod assembly of claim 22, wherein when the plunger rod is in the second position, the plunger rod is rotatable relative to the finger rest in a clockwise direction.

32. The plunger rod assembly of claim 1, further comprising a thumb pad disposed at or adjacent a proximal end of the plunger rod, the thumb pad configured to cause movement of the plunger rod in the axial direction or the first radial direction.

33. A medical device, comprising:
a barrel assembly comprising a barrel adapted to hold a drug product;
the plunger rod assembly of claim 1, wherein the finger rest is coupled to the barrel assembly such that a distal end of the plunger rod is movably disposed in the barrel, and wherein the plunger rod reaches the activated position, the drug product is expelled from the barrel.

34. The medical device of claim 33, wherein the barrel comprises a first chamber and a second chamber, the first chamber adapted to be pre-filled with the drug product, and the second chamber adapted to be pre-filled with a diluent configured to be mixed with the drug product as the plunger rod moves from the first position to the second position.

35. A medical device, comprising:
a barrel comprising a first chamber and a second chamber, the first chamber adapted to be pre-filled with a dry powder of a drug product, and the second chamber adapted to be pre-filled with a diluent configured to be mixed with the dry powder of the drug product;
a finger rest adapted to be coupled to the barrel;
a plunger rod coupled to the finger rest,
wherein the finger rest includes an interface that selectively engages the plunger rod to control movement of the plunger rod relative to the finger rest,
wherein the plunger rod includes a first locking mechanism and a second locking mechanism,
wherein when the plunger rod is in a first position, the first locking mechanism engages a first portion of the interface such that the plunger rod is only movable relative to the finger rest by moving the plunger rod in an axial direction until the plunger rod reaches a second position, thereby causing the diluent to be pushed from the second chamber into the first chamber and preventing inadvertent pre-activation prior to the diluent mixing with the dry powder of the drug product,
wherein when the plunger rod is in the second position, the second locking mechanism engages a second portion of the interface such that the plunger rod is only movable relative to the finger rest by rotating the plunger rod in a first radial direction until the plunger rod reaches a third position, thereby preventing inaccurate activation of the plunger rod, and
wherein when the plunger rod is in the third position, the second locking mechanism engages the first portion of the interface such that the plunger rod is only movable relative to the finger rest by moving the plunger rod in the axial direction until the plunger rod reaches an activated position.

36. The medical device of claim 35, wherein the first locking mechanism comprises a pair of first projections that extend radially outward from opposite sides of the plunger rod.

37. The medical device of claim 35, wherein the second locking mechanism comprises a pair of second projections that extend radially outward from opposite sides of the plunger rod.

38. (canceled)

39. The medical device of claim 35, wherein the plunger rod further comprises a recessed portion that axially separates the first locking mechanism from the second mechanism.

40. The medical device of claim 35, wherein the first portion of the interface comprises a pair of cutouts, wherein the first locking mechanism is disposed in the pair of cutouts when the plunger rod is in the first position, and wherein the second locking mechanism is disposed in the pair of cutouts when the plunger rod is in the third position.

41. The medical device of claim 40, wherein the first locking mechanism is spaced from the pair of cutouts when the plunger rod is in the second position and when the plunger rod is in the third position.

42. The medical device of claim 40, wherein the second locking mechanism is spaced from the pair of cutouts when the plunger rod is in the first position and the plunger rod is in the second position.

43. The medical device of claim 35, wherein the second portion of the interface comprises a pair of shelves, and wherein the second locking mechanism engages the pair of shelves when the plunger rod is in the second position.

44. The medical device of claim 35, wherein the interface comprises a pair of stop surfaces, wherein when the plunger rod is in the second position, the stop surfaces are positioned to prevent rotation of the plunger rod in a second radial direction opposite the first radial direction.

45. (canceled)

46. The plunger rod assembly of claim 1, wherein a length of the second locking mechanism is selected to adjust a volume of headspace in the medical device.

47. The plunger rod assembly of claim 46, wherein the length of the second locking mechanism is selected to increase the volume of headspace in the medical device.

48. The medical device of claim 35, wherein a length of the second locking mechanism is selected to adjust a volume of headspace in the medical device.

49. The medical device of claim 48, wherein the length of the second locking mechanism is selected to increase the volume of headspace in the medical device.
